(19) United States
(12) Patent Application Publication
(10) Pub. No.: US 2017/0198306 A1
(43) Pub. Date: Jul. 13, 2017

(54) ENGINEERING MAMMALIAN GENOME USING DNA-GUIDED ARGONAUTE INTERFERENCE SYSTEMS (DAIS)

(71) Applicant: CELLECTIS, Paris (FR)

(72) Inventors: Julien Valton, New York, NY (US); Philippe Duchateau, Draveil (FR)

(73) Assignee: CELLECTIS, Paris (FR)

(21) Appl. No.: 15/127,128

(22) PCT Filed: Mar. 23, 2015
(86) PCT No.: PCT/EP2015/056113

§ 371 Date: Sep. 19, 2016

(30) Foreign Application Priority Data
Mar. 21, 2014 (DK) PA201470141

(51) Int. Cl. C12N 15/90; C12N 9/22; C12N 15/11; C12N 5/0783; C12N 15/85; C12N 15/86; C12N 5/074

(52) U.S. Cl. CPC C12N 15/907; C12N 15/86; C12N 9/22; C12Y 301/26; C12N 5/0607; C12N 5/0636; C12N 15/8509; C12N 15/111; C12N 2740/15043; C12N 2510/00; C12N 2517/02; C12N 2310/14

(57) ABSTRACT

This invention relates to materials and methods for gene editing in mammalian cells, and more particularly to methods for gene editing using DNA-guided Argonaute (Ago) interference systems (DAIS) in T-cells.

### TECHNICAL FIELD

This patent application relates to materials and methods for gene editing in mammalian cells, and more particularly to methods for gene editing using DNA-guided Argonaute (Ago) interference systems (DAIS) in T-cells.

### BACKGROUND

Argonaute proteins (Ago) from bacteria such as Thermus thermophilus (strain HB27) have been recently described in bacteria to act as a barrier for the uptake and propogation of foreign DNA (Swarts D. C, et al. Nature 507: 258-261) In vivo, Tt Ago is loaded with 5′ phosphorylated DNA guides, from 13 to 25 base pairs that are mostly plasmid derived and have a strong bias for a 5′-end deoxycytidine. These small interfering DNAs guide TtAgo cleave complementary DNA strands at high temperature (75° C.).

On another hand, T-cells are mammalian cells known to be very sensitive to foreign DNA and refractory to DNA transfection.

Here, the inventors surprisingly found that Ago from Thermus thermophilus could be heterologously expressed in mammalian cells and optimized to be active at around 37° C. Based on this finding they have set up a strategy of gene editing using DNA-guided Ago to engineer T-cells suitable for immunotherapy.

### SUMMARY OF INVENTION

As per the present invention, the inventors have established that DNA-guided Argonaute interference system (DAIS) from the prokaryotic bacteria Thermus Thermophilus [1-3] provides an efficient and easy-to-implement tool for generating targeted modifications of genomic DNA. Among them, DAIS can be used for targeted mutagenesis, targeted chromosomal deletions, targeted gene inversion, translocation or insertion and for multiplexed genome modifications. Such technology can be used to engineer living cells for specific applications such as cellular immunotherapy, gene therapy, generation of genetically modified animals, as well as cells for bioproduction as non-limiting examples.

In a more specific aspect, this document presents a method for modifying the genomic material of mammalian cells, especially T-cells. The method includes introducing one, two or multiple short DNA molecules (referred herein as DNA-guides) into the mammalian cells, along with the prokaryotic DAIS that is known to catalyze single strand DNA break at the sequences targeted by DNA-guide. The DAIS can be delivered as DNA, mRNA and purified apo or holo protein (prebound to DNA guide) or via lentivirus. When supplied as DNA, the DAIS coding sequence can be regulated by a constitutive or inducible promoter. The mammalian cells may be primary or immortalized cells, somatic or stem cells including induced Pluripotent Stem Cells (iPSC).

The different features of the invention are detailed in the examples, figures and claims provided hereafter as non-limiting features.

### FIGURES

FIG. 1: Schematic representation of the method for inducing double strand cleavage in a nucleic acid target sequence according to the present invention through the heterologous expression of Ago in a cell in the presence of oligonucleotides that act as specific guides to the selected locus.

FIG. 2: Schematic representation of the method according to the invention using distant cleavage sites, which may either lead to a significant deletion of the locus region (Nx) between the two sites or to a cohesive end cleavage profile.

FIG. 3: Schematic representation showing strategy to inactivate TCR locus in T-cells.

FIG. 4: Schematic representation showing strategy to modify TCR locus in T-cells by homologous recombination using an insertion matrix (donor DNA).

FIGS. 5 and 6: Experimental data (two independent experiments) illustrating that cells treated with DAIS and DNA guides displayed less surface exposed TCR than untransfected cells or cells transfected with DAIS alone. FIG. 5: Dot plots representation of data from experiment 1. FIG. 6: Bar graph representation of data from experiment 2.

### DESCRIPTION

Unless specifically defined herein, all technical and scientific terms used have the same meaning as commonly understood by a skilled artisan in the fields of gene therapy, biochemistry, genetics, and molecular biology.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention broadly relates to a method of modifying the genetic material of a eukaryotic cell, especially an animal cell, and more particularly a mammalian cell through the expression of an Ago protein into said cell in the presence of at least one exogenous oligonucleotide (DNA guide) providing specificity of cleavage to said Ago protein to a preselected locus.

The argonaute (Ago) gene family generally encodes proteins comprising four characteristic domains: N-terminal, PAZ, Mid and a C-terminal catalytic domain referred to as PIWI domain (Meister et al., Molecular Cell 15 (2): 185-197). According to the present invention, Ago proteins refer to any heterologous polypeptide or polynucleotide sequence comprising at least such PIWI domain sequence. Multiple sequence alignment of core motifs of PIWI domains indicate an active site comprising the motif (D/E)-(D/E)XK, X being any standard amino acid. The PIWI domain is believed to contribute to recognition of base pairing with double stranded nucleic acids.

According to a preferred aspect of the invention, the Ago protein has at least 70%, more preferably at least 75%, and even more preferably at least 80%, at least 85%, at least 90%, at least 95%, or at least 99% sequence identity with the Ago protein from prokaryotic bacteria Thermus Thermophilus of SEQ ID NO. 1. (Uniprot database reference Q746M7).

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14 (Basic Local Alignment Search Tool provided by the NCBI at http://ncbi.nlm.nih.gov.) Bl2seq performs a comparison between two sequences using either the BLASTN or BLASTP algorithm. BLASTN is used to compare nucleic acid sequences, while BLASTP is used to compare amino acid sequences.

Optimized Ago proteins can be derived from such a protein, or from an Ago protein from other species, by directed evolution in order to optimize its performance at a range of temperature comprised between 30° C. and 40° C.

One method according to the invention for optimizing an Ago protein to have it induce more cleavage activity at a temperature below 40° C., can comprise the steps of:
a) Introducing oligonucleotides into a cell, said oligonucleotides being selected to hybridize a toxic gene, resistance gene or a reporter gene present into said cell;
b) Creating a variant of the gene encoding Ago protein and expressing said gene into said cell;
c) Cultivating said cell at a temperature below 40° C.;
d) Recovering said variant encoding Ago protein from said cultured cells.
   Such variant proteins obtainable form this method form a further object of the present invention.

According to one aspect of the invention said Ago protein expression may be placed under the control of an inducible promoter to reduce potential genotoxicity of the Ago protein into said cell.

The above method can further comprise the step of cultivating the cells in which cleavage by Ago has occurred at the preselected locus, recovering and isolating said cells in which cleavage by Ago has occurred at the preselected locus. The cells obtained by this method are a further object of the present invention.

The present invention aims more particularly to engineer immune cells, in particular T cells, and most preferably human T cells from patients or donors, for their use in immunotherapy. In particular, the present invention provides with a method for modifying the genetic material of a primary T-cell, comprising at least one of the following steps:
Providing a T-cell from a donor;
Expanding said T-cell;
Transfecting said T-cell with a nucleic acid expressing a Ago Protein;
Further transfecting said T cell with at least one exogenous oligonucleotide (DNA guide) providing specificity of cleavage to said Ago protein to a preselected locus.

Reference is made to pages 28 to 34 of WO2013176915 by the applicant, which are incorporated herein, describing the steps of activating and expanding allogeneic T-cells from donors, which are transduced with nucleic acids, (retroviral or lentiviral vectors or mRNA) encoding chimeric antigen receptors, to result into so-called “CAR immune cells”. This aspect of the invention is illustrated in the following examples with the inactivation of TCR in Jurkat cells and T-cells, in view of providing T-cells from donors that are made “universal”—i.e. suitable for their engraftment into patients, while reducing the risk for graft-versus-host disease.

The oligonucleotide used as a DNA guide in the method according to the present invention is generally 10 to 50 nucleotides in length, preferably 15 to 30 nucleotides, more preferably 20 to 25 nucleotides, which confer a high target specificity to the method of the invention. Such oligonucleotide is preferably phosphorylated at its 5′ terminus, and has also preferably a CA doublet at its 5′ terminus. This is believed to improve the interaction between the guide and the Ago protein.

According to a preferred embodiment of the invention, at least 2 oligonucleotides are selected to respectively hybridize each strand of a double-strand DNA at sites that are closed enough to each other to obtain double strand break. Preferably, the 2 oligonucleotides will be designed to hybridize each strand at the same locus so that Ago will create a blunt double strand break.

According to a further aspect of the invention the method comprises the step of performing homologous recombination at the preselected locus by bringing into the cell a donor DNA comprising a sequence homologous to that of the preselected locus into contact with said genetic material. Various homologous recombination techniques have been described in the prior art, especially in U.S. Pat. No. 6,528,313 and U.S. Pat. No. 8,921,332, but so far have never been practiced using Ago proteins. This method allows donor DNA comprising a transgene, a promoter, an expression cassette or a repairing sequence to be inserted at a preselected locus. Said method can be practiced in gametes and oocytes in view of obtaining cells to develop a transgenic animal.

Otherwise repair mechanism like non homologous end joining (NHEJ) may also be used to introduce transgenes into cell genome upon cleavage by the Ago protein as per the invention.

According to a further aspect of the invention, several oligonucleotides targeting different loci can be carried out to inactivate said loci simultaneously, producing a multiplex genome engineering method. Said additional loci, which may be targeted in said immune cells alone or in combination, are more particularly genes that confer resistance to chemotherapy drugs (ex: fludarabine, chlorofarabine . . . ), such as those genes encoding deoxycytidine kinase (dCk) or encoding hypoxanthine-guanine phosphoribosyl transferase (HPRT) gene thereby conferring resistance to 6-thioguanine (6TG) as described in PCT/EP2014/075317. Resistance to lymphodepleting agents can also be achieved by inactivating certain genes such as those encoding glucocorticoid receptors (GR) and CD52 (target for alemtuzumab).

Further genes may also be inactivated alone or in combination with the previous genes, such as those involved in the expression of major histocompatibility complex (MHC), in particular β2m and HLA genes,

Other genes encoding so-called “immune checkpoints” may also be targeted with the effect of reducing the elimination of the engrafted allogeneic immune-cells by the host's defense system, such as PD1, CTLA4, PPP2CA, PPP2CB, PTPN6, PTPN22, PDCD1, LAG3, HAVCR2, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, MORA, IL1ORB, HMOX2, IL6R, IL6ST, EIF2AK4, CSK, PAG1, SIT1, FOXP3, PRDM1, BATF, GUCY1A2, GUCY1A3, GUCY1B2 and GUCY1B3.

A further aspect of the invention concerns the polynucleotide vectors that are used for the genome engineering of the cells, and the cells transfected with such vectors, prior or after the step of gene inactivation.

According to a preferred embodiment, oligonucleotides are transfected into the cells ex-vivo using electroporation, whereas the polynuclotide encoding the Ago protein is transduced using a retroviral or lentiviral vector. In this regard, the invention encompasses a kit for genetic engineering of cells comprising a polynucleotide encoding Ago protein, preferably introduced into a lentiviral or retroviral vector and at least one oligonucleotide. Another kit is composed of a prokaryotic cell in which the Ago protein is stably introduced, along with at least one oligonucleotides designed to hybridize and inactivate a genomic locus within said cell.

Some applications of the general principles of the invention described above are detailed in the following examples and claims. These examples are not limitative and may be combined with any of the previous aspects of the present invention.

Other features, objects, and advantages of the invention will be apparent from the description and drawings, and from the claims.

### Examples

1—Overall Design and Mechanism of DAIS:

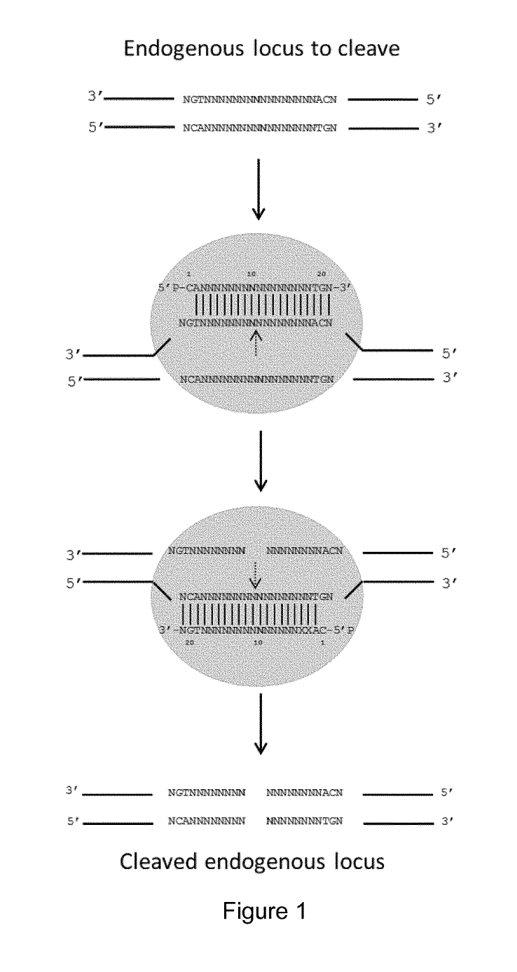

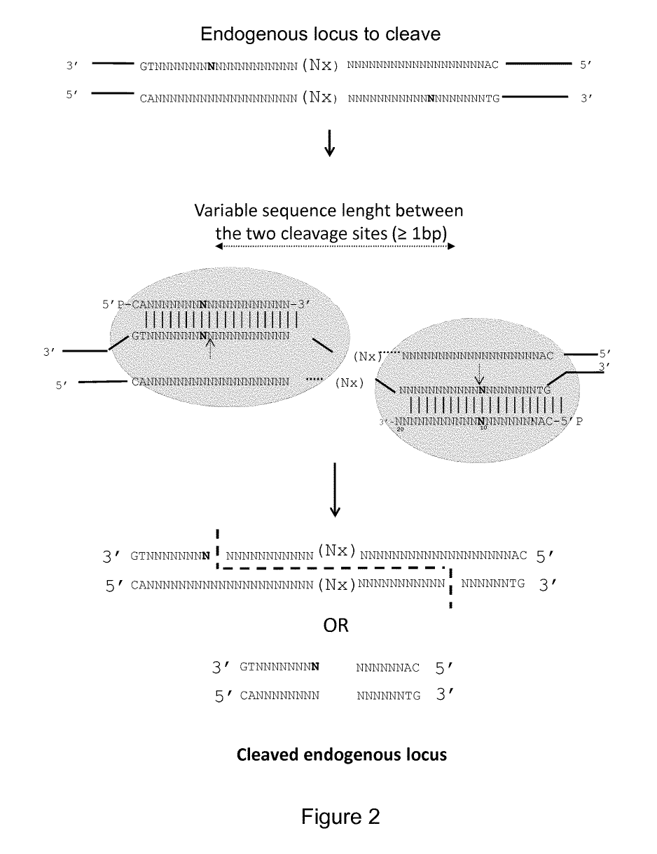

DAIS could be used to process endogenous locus according to different architectures described in FIG. 1. To process an endogenous locus via error-prone NHEJ, DAIS could be used in combination with 2 DNA guide oligonucleotides as illustrated in FIG. 1. DNA guide could be designed to bind to the forward and reverse strand of the locus to process in a complimentary fashion (FIG. 1). They could also be designed to bind to two different DNA targets located on the forward and reverse strand of the locus to process in an uncomplimentary fashion (FIG. 2). Both architectures would catalyze DNA nicking on the reverse and forward strands (FIG. 1, dashed arrows). The nick positions will depend on the DNA guide location. When a given DNA guide is considered, the cleavage is expected to occur between the 10^{th} and the 11^{th} bp of the locus-DNA guide duplex [1,2]. For the sake of clarity, the DNA guide orientation and nucleotide numbering are indicated and the 10^{th} nucleotide is displayed in bold (FIGS. 1 and 2).

2—Heterologous Expression of T. thermophilus Ago in Mammalian Cells:

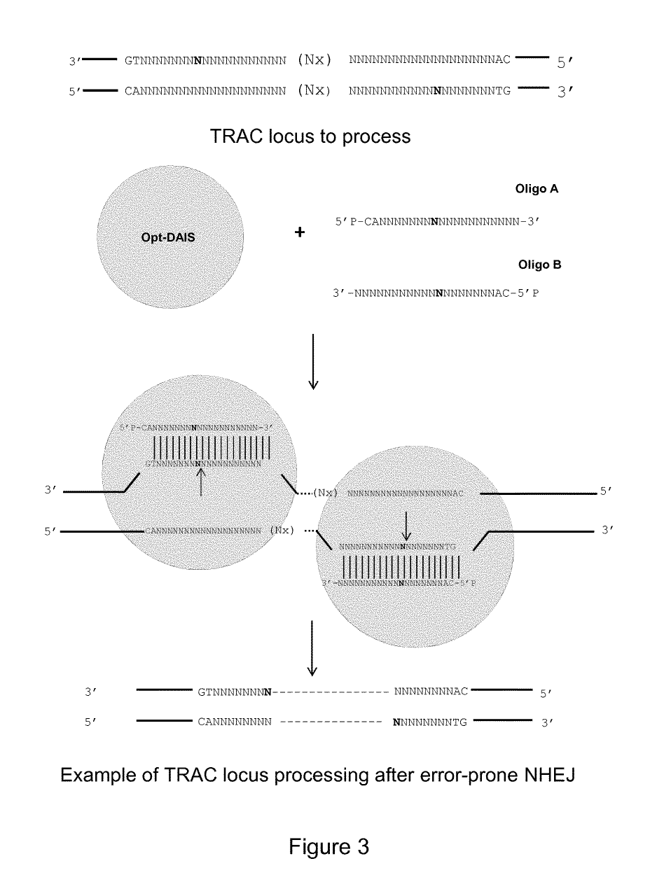

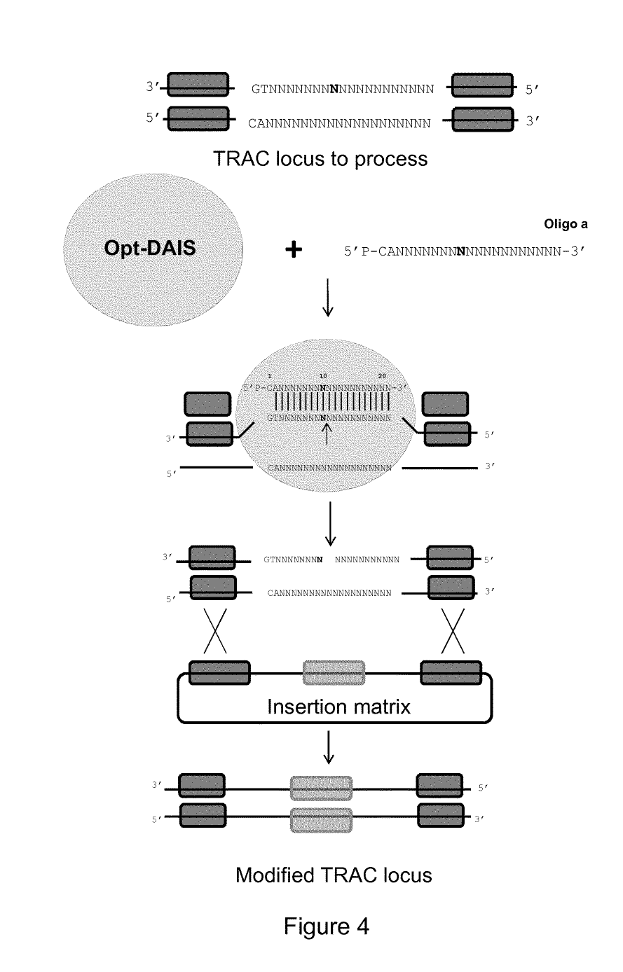

To demonstrate the activity of the DAIS in mammalian cells, a plasmid was constructed to express Thermus thermophilus Argonaute endonucleases (SEQ ID. NO 1) from the HB27 strain (ATCC BAA-163) under the control of pEFlalpha or pCMV promoters in mammalian cells. A second plasmid bearing Thermus thermophilus Argonaute endonuclease coding sequence downstream the pT7 promoter was also constructed to allow in vitro preparation of the corresponding polyadenylated mRNA. A third plasmid bearing Thermus thermophilus Argonaute endonuclease coding sequence linked to the BFP coding sequence via a 2A cis-acting hydrolase element (SEQ ID NO 18 encoding SEQ ID NO 19) and located downstream the pT7 promoter was also constructed to allow in vitro preparation of the corresponding polyadenylated mRNA. A fourth plasmid, enabling the overexpression and purification of homogenous preparation of Thermus thermophilus Argonaute endonuclease bearing N- or C-term affinity tag (for purification purpose), was also prepared. Different DNA guides oligonucleotides (SEQ ID NO 6-17) complementary to the forward and reverse strand of the TRAC locus (FIG. 3) were chemically synthesized. Each DNA guide oligonucleotide consisted in 21 bp DNA oligonucleotide harboring a 5′ phosphate group.

3—DAIS Endonucleases Activity in Jurkat T Cells:

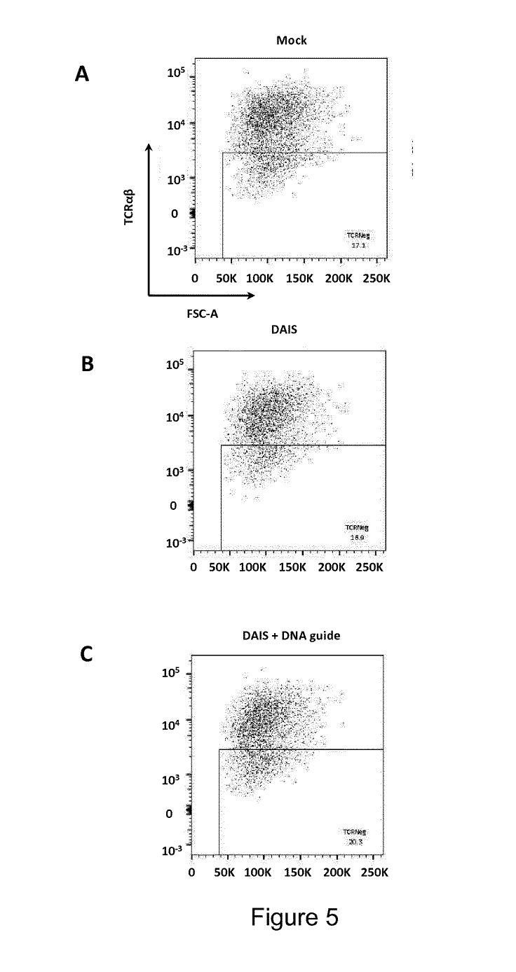

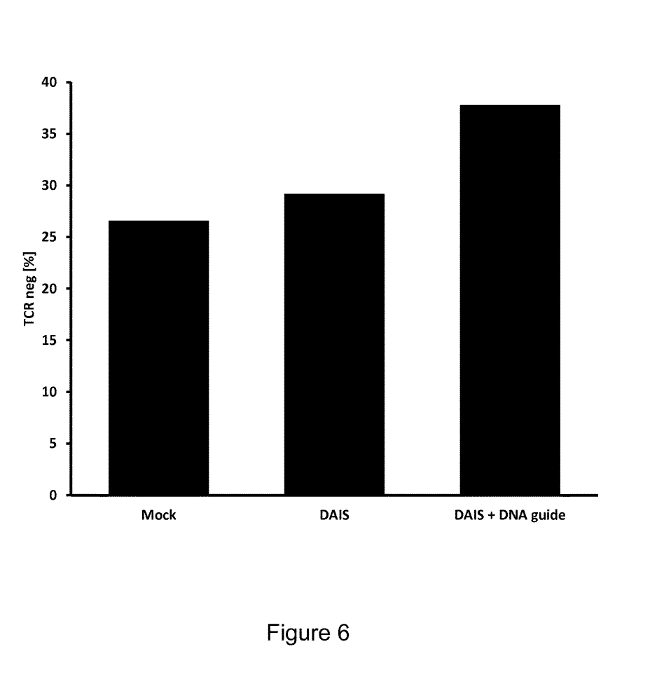

To test the ability of DAIS to promote error-prone NHEJ events at the TRAC locus (FIG. 3), 20 μg of mRNA encoding DAIS-2A-BFP (SEQ ID NO 19) were electroporated in the presence of 2 DNA guides chosen in the list described in table I (SEQ ID NO 6-17) in Primary T cells using Pulse Agile technology according to the manufacturer protocol. One day post transfection, cells were analyzed by flow cytometry to verify expression of the BFP linked to the Argonaute coding sequence via a 2A-cis acting hydrolase element. Results showed a significant expression of BFP suggesting efficient expression of Argonaute protein. Six days post transfection, cells were analysed by flow cytometry to determine the amount of surface exposed TCR remaining after the argonaute treatment. In parallel genomic DNA were extracted from treated cells to perform a PCR amplification of the TRAC locus. The resulting amplicon was subjected to Endo T7 assay to determine the extent of targeted mutagenesis promoted by the DAIS at the TRAC locus. Our results showed that cells treated with DAIS and DNA guides displayed less surface exposed TCR than untransfected cells or cells transfected with DAIS alone (FIGS. 5 and 6). These results were consistent with a detectable endo T7 signal obtained with cells transfected with DAIS along with 2 DNA guides and with the absence of signal obtained with untransfected cells or cells transfected with DAIS alone. This indicates that DAIS is able to promote error-prone NHEJ at the TRAC locus.

TABLE 1
Polypeptide and polynucleotide sequences used for
inactivating TCRα gene in human T- cells

| | SEQ ID | Polypeptide/Polynucleotide |
|---|---|---|
| Name | # | sequences 5′ > 3′ |
| Thermus | SEQ ID | MNHLGKTEVFLNRFALRPLNPEELRPWR |
| thermophilus | NO. 1 | LEVVLDPPPGREEVYPLLAQVARRAGGV |
| Argonaute | | TVRMGDGLASWSPPEVLVLEGTLARMGQ |
| | | TYAYRLYPKGRRPLDPKDPGERSVLSAL |
| | | ARRLLQERLRRLEGVWVEGLAVYRREHA |
| | | RGPGWRVLGGAVLDLWVSDSGAFLLEVD |
| | | PAYRILCEMSLEAWLAQGHPLPKRVRNA |
| | | YDRRTWELLRLGEEDPKELPLPGGLSLL |
| | | DYHASKGRLQGREGGRVAWVADPKDPRK |
| | | PIPHLTGLLVPVLTLEDLHEEEGSLALS |
| | | LPWEERRRRTREIASWIGRRLGLGTPEA |
| | | VRAQAYRLSIPKLMGRRAVSKPADALRV |
| | | GFYRAQETALALLRLDGAQGWPEFLRRA |
| | | LLRAFGASGASLRLHTLHAHPSQGLAFR |
| | | EALRKAKEEGVQAVLVLTPPMAWEDRNR |
| | | LKALLLREGLPSQILNVPLREEERHRWE |
| | | NALLGLLAKAGLQVVALSGAYPAELAVG |
| | | FDAGGRESFRFGGAACAVGGDGGHLLWT |
| | | LPEAQAGERIPQEVVWDLLEETLWAFRR |
| | | KAGRLPSRVLLLRDGRVPQDEFALALEA |
| | | LAREGIAYDLVSVRKSGGGRVYPVQGRL |
| | | ADGLYVPLEDKTFLLLTVHRDFRGTPRP |
| | | LKLVHEAGDTPLEALAHQIFHLTRLYPA |
| | | SGFAFPRLPAPLHLADRLVKEVGRLGIR |
| | | HLKEVDREKLFFV |
| TRAC | SEQ ID | CACAAAGTAAGGATTCTGATGTGTATAT |
| target 1 | NO. 2 | CACAGACAAAACTG |
| TRAC | SEQ ID | CATGAGGTCTATGGACTTCAAGAGCAAC |
| target 2 | NO. 3 | AGTGCTGTGGCCTG |
| TRAC | SEQ ID | CAAAGTAAGGATTCTGATGTGTATATCA |
| target 3 | NO. 4 | CAGACAAAACTGTG |
| TRAC | SEQ ID | CCACAGATATCCAGAACCCTGA |
| target 7 | NO. 5 | |
| TRAC | SEQ ID | CACAAAGTAAGGATTCTGATG |
| Oligo a | NO. 6 | |
| target 1 | | |
| TRAC | SEQ ID | CAGTTTTGTCTGTGATATACA |
| Oligo b | NO. 7 | |
| target 1 | | |
| TRAC | SEQ ID | CATGAGGTCTATGGACTTCAA |
| Oligo a | NO. 8 | |
| target 2 | | |
| TRAC | SEQ ID | CAGGCCACAGCACTGTTGCTC |
| Oligo b | NO. 9 | |
| target 2 | | |
| TRAC | SEQ ID | CAAAGTAAGGATTCTGATGTG |
| Oligo a | NO. 10 | |
| target 3 | | |
| TRAC | SEQ ID | CACAGTTTTGTCTGTGATATA |
| Oligo b | NO. 11 | |
| target 3 | | |
| TRAC | SEQ ID | CACAGATATCCAGAACCCTGA |
| Oligo a | NO. 12 | |
| target 7 | | |
| TRAC | SEQ ID | CAGGGTTCTGGATATCTGTGG |
| Oligo b | NO. 13 | |
| target 7a | | |
| TRAC | SEQ ID | CAGCTGGTACACGGCAGGGTC |
| Oligo b | NO. 14 | |
| target 7b | | |
| TRAC | SEQ ID | CACTGGATTTAGAGTCTCTCA |
| Oligo b | NO. 15 | |
| target 7c | | |
| TRAC | SEQ ID | CACGGCAGGGTCAGGGTTCTG |
| Oligo b | NO. 16 | |
| target 7d | | |
| TRAC | SEQ ID | CAGGGTCAGGGTTCTGGATAT |
| Oligo b | NO. 17 | |
| target 7e | | |

4—DAIS Endonucleases Activity in Primary T Cells:

To test the ability of DAIS to promote error-prone NHEJ events at the TRAC locus (FIG. 3), 20 μg of mRNA encoding DAIS were electroporated in the presence of 2 DNA guides chosen in the list described in table I (SEQ ID NO 6-17) in Primary T cells using Pulse Agile technology according to the manufacturer protocol (Harvard Apparatus, Holliston, Mass. 01746, USA). Six days post transfection, cells were recovered and genomic DNA was extracted. PCR amplification of TRAC endogenous locus was then performed and the resulting amplicon was subjected to Endo T7 assay to determine the extent of targeted mutagenesis promoted by the DAIS at the TRAC locus. Our results showed a barely detectable endo T7 signal, indicating that DAIS is able to promote error-prone NHEJ at the TRAC locus although with a low efficiency.

5—Random Mutagenesis of DAIS to Improve its Nuclease Activity in Primary T Cells:

To improve the catalytic efficiency of DAIS at temperature suitable to mammalian cell culture, the DNA sequence encoding the DAIS (SEQ ID NO 1) was subjected to random mutagenesis. Such process excluded I434, Q433, K422, K457, D478, E512, D546 and D660 and their proximal neighboring amino acids, reported to play a key role either in the DNA binding and/or catalytic mechanism of DAIS [1]. The resulting library of mutated DAIS was transformed in bacteria and then screened for its ability to disrupt a toxic gene at 37° C. Bacterial transformants that were able to grow at 37° C. were recovered and their plasmidic DNA content was extracted. The resulting DNA sequence encoding the optimized DAIS (Opt-DAIS) was then used to assess the endonucleases activity of the system in primary T cells according to the experimental protocol described in Example 2. Our results showed that the optimized Opt-DAIS display a higher nuclease activity than the wild type version at the locus considered.

6—Harnessing Opt-DAIS Endonuclease Activity to Promote Homologous Gene Targeting (HGT) in Primary T Cells:

To demonstrate the ability of Opt-DAIS to promote Homologous gene targeting (HGT) at the TRAC locus, 5 or 10 μg of mRNA encoding Opt-DAIS were electroporated in T cells in the presence of one or multiple DNA guides belonging to list described in earlier examples (SEQ ID NO 6-13) and of a linearized plasmidic DNA insertion matrix specifically designed to promote HGT at the TRAC locus. Briefly, the insertion matrices encompassed a 50 bp exogenous DNA sequence flanked by two 500 bp homology sequences (the left and right homology sequences) identical to the targeted locus considered. Alternatively the DNA matrix could be supplied as modified ssDNA or dsDNA oligonucleotide. Such matrix typically consists in a 50 bp exogenous sequence flanked by two 100 bp left and right homology sequences. To prevent its recruitment and utilization by DAIS as DNA guide, the matrix could be lacking the 5′end phosphate moiety and could harbors one or multiple chemical groups designed and positioned to successfully prevent binding with Opt-DAIS.

Cells were recovered 3 days post transfection, genomic DNA was extracted and used to perform HGT-specific PCR screening and determine the ability of Opt-DAIS to promote HGT. Our result showed some HGT positive PCR band indicating that Opt-DAIS was able to promote HGT in primary T cells.

### REFERENCES

1. Sheng G, Zhao H, Wang J, Rao Y, Tian W, et al. (2014) Structure-based cleavage mechanism of Thermus thermophilus Argonaute DNA guide strand-mediated DNA target cleavage. Proc Natl Acad Sci USA 111: 652-657.
2. Swarts D C, Jore M M, Westra E R, Zhu Y, Janssen J H, et al. (2014) DNA-guided DNA interference by a prokaryotic Argonaute. Nature 507: 258-261.
3. Makarova K S, Wolf Y I, van der Oost J, Koonin E V. (2009) Prokaryotic homologs of Argonaute proteins are predicted to function as key components of a novel system of defense against mobile genetic elements. Biol Direct 4: 29.

1. A method of modifying the genetic material of an animal cell through expression of an Ago protein into said cell in the presence of at least one exogenous oligonucleotide (DNA guide) providing specificity of cleavage to said Ago protein to a preselected locus.

2. The method according to claim 1, further comprising the step of cultivating the cells in which cleavage by Ago has occurred at the preselected locus.

3. The method according to claim 2, further comprising the step of recovering the culture supernatant of said cultured cells to recover the molecules produced by the modified cells.

4. The method according to claim 1, further comprising the step of recovering the cells in which cleavage by Ago has occurred at the preselected locus.

5. The method according to claim 1, further comprising the step of freezing or conditioning the cells in which cleavage by Ago has occurred at the preselected locus, for use as a therapeutic product.

6. The method according to claim 1, wherein said cell is a mammalian cell.

7. The method according to claim 1, wherein said cell is a human cell.

8. The method according to claim 1, wherein said cell is a T-cell.

9. The method according to claim 8, wherein said locus in said T-cell is selected from the genes encoding T cell receptor (TCR), Glucocorticoid receptors (GR), dCK, β2m, HLA, HPRT, PD1, CTLA4, PPP2CA, PPP2CB, PTPN6, PTPN22, PDCD1, LAG 3, HAVCR2, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1 B2 and GUCY1B3.

10. The method according to claim 1, wherein said oligonucleotide is 10 to 50 nucleotides in length, preferably 15 to 30 nucleotides, more preferably 20 to 25 nucleotides.

11. The method according to claim 1, wherein said oligonucleotide is phosphorylated, preferably at its 5′ terminus.

12. The method according to claim 1, wherein said oligonucleotide has a CA doublet at its 5′ terminus.

13. The method according to claim 1, wherein several loci are cleaved using various specific oligonucleotides (multiplex).

14. The method according to claim 1, wherein at least 2 oligonucleotides are selected to respectively hybridize each strand of a double-strand DNA at sites that are closed enough to each other to obtain double strand break.

15. The method according to claim 1, wherein the 2 oligonucleotides hybridize each strand at the same locus so that Ago will create a blunt double strand break.

16. The method according to claim 1, further comprising the step of performing homologous recombination at the preselected locus by bringing a donor DNA comprising a sequence homologous to that of the preselected locus into contact with said genetic material.

17. The method according to claim 16, wherein said donor DNA comprises a transgene, a promoter, an expression cassette or a repairing sequence to be inserted at the preselected locus.

18. The method according to claim 1, wherein said Ago protein is heterologously expressed from a polynucleotide introduced into said cell.

19. The method according to claim 1, wherein said Ago protein has at least 70% sequence identity with SEQ ID NO. 1.

20. The method according to claim 1, wherein said Ago protein is optimized to be more active at a temperature below 40° C.

21. The method according to claim 1, wherein said Ago protein is optimized to be more active at a temperature between 30 and 40° C., preferably 37° C.

22. The method according to claim 18, wherein said polynucleotide encoding said Ago protein is transduced by a retroviral or lentiviral vector.

23. The method according to claim 18, wherein said polynucleotide is mRNA.

24. The method according to claim 18, wherein said mRNA is introduced into said cell by electroporation.

25. The method according to claim 1, wherein said Ago protein expression is under the control of an inducible promoter to reduce potential genotoxicity of the Ago protein into said cell.

26. The method according to claim 1, further comprising the step of introducing said modified genetic material into an animal stem cell to develop a transgenic animal.

27. A polynucleotide vector comprising a gene encoding Ago protein.

28. A retrovirus or lentiviral vector comprising a polynucleotide encoding an Ago protein for transducing mammalian cells.

29. A kit for genetic engineering of cells comprising a polynucleotide encoding Ago protein and at least one oligonucleotide.

30. A method for optimizing Ago protein to induce more cleavage activity at a temperature below 40° C., wherein said method comprises the following steps:
a) Introducing oligonucleotides into a cell, said oligonucleotides being selected to hybridize a toxic gene, resistance gene or a reporter gene present into said cell;
b) Creating a variant of the gene encoding Ago protein and expressing said gene into said cell;
c) Cultivating said cell at a temperature below 40° C.;
d) Recovering said variant encoding Ago protein from said cultured cells that do not express said toxic gene, resistance gene or reporter gene.

31. The method according to claim 30, wherein said Ago protein has at least 70% sequence identity with SEQ ID NO. 1.

32. The method according to claim 30, wherein said temperature is between 30 and 40° C.

33. The method according to claim 30, wherein said cell is a mammalian cell.

34. The method according to claim 30, wherein said Ago protein is assayed for more stable expression in said cell at said temperature.

35. An optimized variant gene encoding Ago protein obtained by a method of comprising the following steps:
a) Introducing oligonucleotides into a cell, said oligonucleotides being selected to hybridize a toxic gene, resistance gene or a reporter gene present into said cell;
b) Creating a variant of the gene encoding Ago protein and expressing said gene into said cell:
c) Cultivating said cell at a temperature below 40° C.; and
d) Recovering said variant encoding Ago protein from said cultured cells that do not express said toxic gene, resistance gene or reporter gene.

36. An ago protein encoded by a variant gene obtained by a method comprising the following steps:
a) Introducing oligonucleotides into a cell, said oligonucleotides being selected to hybridize a toxic gene, resistance gene or a reporter gene present into said cell;
b) Creating a variant of the gene encoding Ago protein and expressing said gene into said cell;
c) Cultivating said cell at a temperature below 40° C.; and
d) Recovering said variant encoding Ago protein from said cultured cells that do not express said toxic gene, resistance gene or reporter gene.


SEQUENCE LISTING (19 sequences)

SEQ ID NO: 1 - protein, 685 aa, Thermus thermophilus
MNHLGKTEVFLNRFALRPLNPEELRPWRLEVVLDPPPGREEVYPLLAQVARRAGGVTVRMGDGLASWSPPEVLVLEGTLARMGQTYAYRLYPKGRRPLDPKDPGERSVLSALARRLLQERLRRLEGVWVEGLAVYRREHARGPGWRVLGGAVLDLWVSDSGAFLLEVDPAYRILCEMSLEAWLAQGHPLPKRVRNAYDRRTWELLRLGEEDPKELPLPGGLSLLDYHASKGRLQGREGGRVAWVADPKDPRKPIPHLTGLLVPVLTLEDLHEEEGSLALSLPWEERRRRTREIASWIGRRLGLGTPEAVRAQAYRLSIPKLMGRRAVSKPADALRVGFYRAQETALALLRLDGAQGWPEFLRRALLRAFGASGASLRLHTLHAHPSQGLAFREALRKAKEEGVQAVLVLTPPMAWEDRNRLKALLLREGLPSQILNVPLREEERHRWENALLGLLAKAGLQVVALSGAYPAELAVGFDAGGRESFRFGGAACAVGGDGGHLLWTLPEAQAGERIPQEVVWDLLEETLWAFRRKAGRLPSRVLLLRDGRVPQDEFALALEALAREGIAYDLVSVRKSGGGRVYPVQGRLADGLYVPLEDKTFLLLTVHRDFRGTPRPLKLVHEAGDTPLEALAHQIFHLTRLYPASGFAFPRLPAPLHLADRLVKEVGRLGIRHLKEVDREKLFFV

SEQ ID NO: 2 - DNA, 42 bp, artificial sequence
  description of artificial sequence synthetic polynucleotide
cacaaagtaaggattctgatgtgtatatcacagacaaaactg

SEQ ID NO: 3 - DNA, 42 bp, artificial sequence
  description of artificial sequence synthetic polynucleotide
catgaggtctatggacttcaagagcaacagtgctgtggcctg

SEQ ID NO: 4 - DNA, 42 bp, artificial sequence
  description of artificial sequence synthetic polynucleotide
caaagtaaggattctgatgtgtatatcacagacaaaactgtg

SEQ ID NO: 5 - DNA, 22 bp, artificial sequence
  description of artificial sequence synthetic polynucleotide
ccacagatatccagaaccctga

SEQ ID NO: 6 - DNA, 21 bp, artificial sequence
  description of artificial sequence synthetic polynucleotide
cacaaagtaaggattctgatg

SEQ ID NO: 7 - DNA, 21 bp, artificial sequence
  description of artificial sequence synthetic polynucleotide
cagttttgtctgtgatataca

SEQ ID NO: 8 - DNA, 21 bp, artificial sequence
  description of artificial sequence synthetic polynucleotide
catgaggtctatggacttcaa

SEQ ID NO: 9 - DNA, 21 bp, artificial sequence
  description of artificial sequence synthetic polynucleotide
caggccacagcactgttgctc

SEQ ID NO: 10 - DNA, 21 bp, artificial sequence
  description of artificial sequence synthetic polynucleotide
caaagtaaggattctgatgtg

SEQ ID NO: 11 - DNA, 21 bp, artificial sequence
  description of artificial sequence synthetic polynucleotide
cacagttttgtctgtgatata

SEQ ID NO: 12 - DNA, 21 bp, artificial sequence
  description of artificial sequence synthetic polynucleotide
cacagatatccagaaccctga

SEQ ID NO: 13 - DNA, 21 bp, artificial sequence
  description of artificial sequence synthetic polynucleotide
cagggttctggatatctgtgg

SEQ ID NO: 14 - DNA, 21 bp, artificial sequence
  description of artificial sequence synthetic polynucleotide
cagctggtacacggcagggtc

SEQ ID NO: 15 - DNA, 21 bp, artificial sequence
  description of artificial sequence synthetic polynucleotide
cactggatttagagtctctca

SEQ ID NO: 16 - DNA, 21 bp, artificial sequence
  description of artificial sequence synthetic polynucleotide
cacggcagggtcagggttctg

SEQ ID NO: 17 - DNA, 21 bp, artificial sequence
  description of artificial sequence synthetic polynucleotide
cagggtcagggttctggatat

SEQ ID NO: 18 - DNA, 2820 bp, Artificial Sequence
  DAIS-2A-BFP
atgaatcacctgggcaagaccgaggtgttcctgaacaggttcgccctcaggccactgaaccccgaggagctccggccctggaggctcgaagtggtgctcgacccaccccctggaagagaggaggtgtaccccctgctcgcccaggtcgcaaggagagccggaggagtgaccgtcagaatgggggacggactggcatcctggagcccacctgaggtgctggtgctggagggaaccctggcaagaatggggcagacctacgcctacaggctctaccccaaggggcgcagacccctcgaccctaaggaccctggggagagaagcgtgctctccgcactcgcaaggagactgctccaggagcggctcaggagactggagggagtgtgggtggagggactcgcagtctacaggagggagcacgcaaggggccctggatggagagtcctcggaggagcagtgctcgacctctgggtgtccgattccggggcattcctgctggaggtggaccctgcttaccgcatcctgtgcgagatgtccctggaggcctggctcgcccagggacaccctctccctaagcgggtgagaaacgcctacgaccggagaacctgggagctcctccggctgggagaggaagatcccaaggagctccccctgcctggagggctcagcctcctcgactaccacgcttccaaggggagactgcaggggagagaggggggcagagtcgcttgggtcgctgaccctaaagaccctagaaagccaatcccccacctcaccgggctgctggtgccagtcctgaccctggaggacctccacgaggaggaaggctctctggctctcagcctcccttgggaggagaggcgccgcagaacaagggagatcgcctcctggatcggcaggaggcttggccttgggacaccagaggctgtgagggctcaggcttaccggctctccatcccaaagctgatggggcggagggctgtgagcaagcccgctgacgctctgagggtcgggttctacagggctcaggaaacagccctggccctcctgaggctggacggggctcaagggtggccagagttcctgaggagggccctgctgagggctttcggcgcttctggcgcctcacttaggctgcacacactgcacgcccacccatctcagggccttgccttcagggaggccttgaggaaggccaaggaggagggggtgcaggccgtccttgtcttgactcctcctatggcctgggaggacaggaacaggctgaaggccctgcttcttagggaggggttgccaagccagattctgaacgtgcccctgcgggaggaagaacggcatcggtgggagaacgccctgctgggcttgctggccaaagccgggcttcaagtggtggccttgagcggggcctatccagccgaactggccgtcgggtttgatgccggcggccgggaatcatttcgcttcggcggggccgcctgtgccgtggggggggatggcggccatttgttgtggacacttccagaagcccaggccggcgaacggattccacaggaagtcgtgtgggatttgcttgaggagactctgtgggccttccgccggaaagccggccggttgcccagtcgggtcttgttgcttcgggatgggcgggtgccacaggatgagtttgccttggccttggaagcccttgcccgcgaggggattgcctatgatctggtgagcgtgcggaagagcgggggcggccgggtctaccccgtccagggccggttggccgatggcttgtacgtgcccttggaggataaaactttcttgctgctgacagtgcaccgggattttcggggcactccccgccccctgaagctggtccacgaggccggcgatacacccctggaggccctggcccatcagatttttcacctgactcggctgtaccccgccagtggctttgcctttccccggctgcccgcccccctgcatctggccgatcggctggtcaaggaggtcggccggctgggcatccggcatctgaaggaggtggatcgggagaagctgttttttgtgggatccgagggcagaggcagcctgctgacctgcggcgacgtggaggagaacccaggcccctccggaagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagctcaattaa

SEQ ID NO: 19 - protein, 939 aa, Artificial Sequence
  DAIS-2A-BFP
MNHLGKTEVFLNRFALRPLNPEELRPWRLEVVLDPPPGREEVYPLLAQVARRAGGVTVRMGDGLASWSPPEVLVLEGTLARMGQTYAYRLYPKGRRPLDPKDPGERSVLSALARRLLQERLRRLEGVWVEGLAVYRREHARGPGWRVLGGAVLDLWVSDSGAFLLEVDPAYRILCEMSLEAWLAQGHPLPKRVRNAYDRRTWELLRLGEEDPKELPLPGGLSLLDYHASKGRLQGREGGRVAWVADPKDPRKPIPHLTGLLVPVLTLEDLHEEEGSLALSLPWEERRRRTREIASWIGRRLGLGTPEAVRAQAYRLSIPKLMGRRAVSKPADALRVGFYRAQETALALLRLDGAQGWPEFLRRALLRAFGASGASLRLHTLHAHPSQGLAFREALRKAKEEGVQAVLVLTPPMAWEDRNRLKALLLREGLPSQILNVPLREEERHRWENALLGLLAKAGLQVVALSGAYPAELAVGFDAGGRESFRFGGAACAVGGDGGHLLWTLPEAQAGERIPQEVVWDLLEETLWAFRRKAGRLPSRVLLLRDGRVPQDEFALALEALAREGIAYDLVSVRKSGGGRVYPVQGRLADGLYVPLEDKTFLLLTVHRDFRGTPRPLKLVHEAGDTPLEALAHQIFHLTRLYPASGFAFPRLPAPLHLADRLVKEVGRLGIRHLKEVDREKLFFVGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN